(19) United States
(12) Patent Application Publication
(10) Pub. No.: US 2021/0375455 A1
(43) Pub. Date: Dec. 2, 2021

(54) ARTIFICIAL INTELLIGENCE SYSTEMS THAT INCORPORATE EXPERT KNOWLEDGE RELATED TO HEART FAILURE

(71) Applicant: MediSync, Inc., Cincinnati, OH (US)

(72) Inventors: Robert E. Matthews, Cincinnati, OH (US); Charlie Hardtke, Cincinnati, OH (US); Douglas Romer, Cincinnati, OH (US); Todd O'Connell, Cincinnati, OH (US)

(21) Appl. No.: 16/885,034

(22) Filed: May 27, 2020

(51) Int. Cl. G16H 50/20; G16H 10/60; G16H 20/10; G06N 5/04; G06N 5/02; G16B 5/00; A61B 5/024; A61K 31/18

(52) U.S. Cl. CPC G16H 50/20; G16H 10/60; G16H 20/10; A61K 31/18; G06N 5/025; G16B 5/00; A61B 5/024; G06N 5/04

(57) ABSTRACT

Provided is a process that includes: obtaining, with an artificial intelligence (AI) application executed by a computer system, information about a current patient condition related to heart failure; inputting, with the computer system, at least some of the obtained information about the patient condition into respective pharmaceutical-specific models to obtain candidate classes of pharmaceuticals and their respective priority scores; inputting, with the computer system, into an expert system of the AI application, the candidate classes of pharmaceuticals and their respective priority scores and, in response, determining an updated prescription.

### CROSS-REFERENCE TO RELATED APPLICATIONS

No cross-reference is presented at this time.

### BACKGROUND

### 1. Field

The present disclosure relates generally to computer systems to aid medical professionals and, more specifically, to artificial intelligence systems that incorporate expert knowledge related to heart failure.

### 2. Description of the Related Art

Physicians and other medical practitioners contend with an enormous amount of complexity when treating patients. A significant amount of that complexity is involved in selecting the medications that can best help a specific patient and that patient's vast array of specific variables. Often, doctors have to make decisions under uncertainty based upon relatively noisy, high dimensional data about patients, and those signals can evolve over time, and some cases in ways that are difficult to predict.

Medical practitioners are trained in and are taught how to apply medical research to select the appropriate interventions for patients. However, at this point in time there are approximately 800,000 new medical journal articles published per year and the amount of published data has grown beyond a human's ability to read, recall or apply in real world clinical practice.

In order to win Food and Drug Administration (FDA) approval every medication must be tested in studies that are carefully constructed and executed under FDA supervision and approval. Subsequent studies of the same drug may occur under independent auspices or sponsored by the drug manufacturer. Each study has a certain set of inclusionary and exclusionary variables. Different studies of the same drug can and often do have different inclusion and exclusion variables and values. When physicians go to prescribe a drug, it is unlikely that they can recall from memory which studies investigated that condition or that drug and extremely unlikely that to recall any of the inclusion and exclusion variables. Thus, determining which drug to use is beyond difficult. Many physicians treat a great number of medical diagnoses thus raising the amount of memory and complexity to an impossible or, at least, high unlikely level.

To assist those in the field, on occasion teams of experts are convened under the authority of different national or international authorities like the American College of Cardiology, the American Heart Association, the Institute of Medicine, etc. to sort out the various studies related to a specific disease state and to update the science that is available with regard to that disease state concluding with high level findings. After hundreds of thousands of pages of published scientific papers, for example, a body might establish a standard of care sometimes called “The Evidence Based Standard of Care” (EBS). At a minimum an EBS or another standard will define, in some fields, a standard by which successful therapy or treatment can be measured, e.g., a goal for therapy and general comments about a variety of tools one could use to achieve that goal. But these standards can be challenging to implement, become out of date, or apply less granular heuristics to favor administrability over precision.

Given these challenges, only very recently have developers and computer science researchers begun to attempt to help doctors choose the appropriate course of treatments for patients, and many of these attempts to date have been limited to diseases like cancer. These attempts, however, have not been met with success generally. Some approaches have attempted to apply expert systems that encode the universe of medical knowledge in a collection of rules. The problem with rules is that the number of variables combined with the way that variables can interact and the number of solution options can approach tens of millions of permutations, which is beyond the capability for rule making. In general, expert systems have proven too brittle, i.e., unable to generalize outside of scenarios explicitly contemplated by the system architect. Such systems often struggle with patients presenting novel scenarios, data entry errors, and variations in data schemas. On the other hand, some researchers have attempted to train models with relatively large numbers of degrees of freedom, like deep neural networks, on historical records of treatments and patient responses. These systems, however, often fail to benefit from the knowledge produced by medical research and in many cases have a relatively low training efficiency. These models struggle with smaller training sets and training sets in which samples are sparse in areas in which the model may be later requested to perform. There is also the problem that medical data is often extremely noisy and filled with error.

### SUMMARY

The following is a non-exhaustive listing of some aspects of the present techniques. These and other aspects are described in the following disclosure.

Some aspects include a process, including: obtaining, with an artificial intelligence (AI) application executed by a computer system, information about a current patient condition, wherein: the information relates to a patient who has undergone heart failure, at least some of the information is obtained from an electronic medical records system via an application program interface of the electronic medical records system, at least some of the information is validated or entered into the AI application by a medical professional after obtaining information from the electronic medical records system, and the AI application comprises: a plurality of pharmaceutical-specific models each corresponding to a different class of pharmaceuticals to treat heart failure and each configured to predict a response of the patient to a change in dosage of the corresponding class of pharmaceuticals given the current patient condition, and an expert system comprising a plurality of rules in a rule-graph, the rule-graph being traversable to determine an updated prescription based on the patient condition and the predicted patient responses; inputting, with the computer system, at least some of the obtained information about the patient condition into the respective pharmaceutical-specific models and, in response: predicting respective changes in the condition of the patient responsive to respective changes in respective dosages of the respective classes of pharmaceuticals to obtain a set of predicted patient responses for the different classes of pharmaceutical, selecting based on the set of predicted patient responses, candidate classes of pharmaceuticals for an updated prescription, determining differences between target dosages and the current dosages of the classes of pharmaceuticals, and based on the differences, determining respective priority scores of the respective classes of pharmaceuticals; inputting, with the computer system, into the expert system of the AI application, the candidate classes of pharmaceuticals and their respective priority scores and, in response, determining an updated prescription; and storing, with the computer system, the updated prescription in memory.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a logical architecture block diagram that illustrates an example of a heart-failure artificial intelligence (AI) application consistent with some embodiments of the present techniques;

FIG. 2 is a flowchart that illustrates an example of a process performed by the system of FIG. 1 consistent with some embodiments of the present techniques;

FIG. 3 illustrates a first portion of a rule-graph consistent with some embodiments of the present techniques;

FIG. 4 illustrates a second portion of the rule-graph consistent with some embodiments of the present techniques;

FIG. 5 illustrates a third portion of the rule-graph consistent with some embodiments of the present techniques; and

FIG. 6 is a block diagram of an example of a computer system upon which some embodiments of the present techniques may execute.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the fields of computer science, AI, and human-computer interaction. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

The issues discussed above are particularly acute when dealing with the aftermath of heart failure of a patient. There are evidence-based standards that specify a variety of different types of treatments to be applied based upon different neural hormones released in the body that cause or increase the damage in cardiac muscle that is called heart failure. Treatment regimens, however, as specified in the standards, are enormously complex. Physicians have a variety of different medications to choose from, different medications are suitable for different symptoms, and interactions between medications and various morbidities are complex. Typically, physicians implement a multi-step process to gradually ramp up different classes of pharmaceuticals while monitoring for various undesirable phenomena relating to things like heart rate, systolic and diastolic pressure, kidney function, potassium levels, and the like that can reach undesirable levels if not handled with care during the process. Often, physicians gradually incrementally adjust the different pharmaceuticals, and some cases decreasing dosages or switch between different classes of drugs, responsive to feedback from lab results and observations of the patient, in many cases over 5, 10, 15, or more increments spanning days, weeks, months, or longer.

As a result of this complexity, physicians struggle with adhering to evidence-based standards. Studies have documented a four-year survival rate following diagnosis with heart failure of around 50%, yet it has been documented that certain medications recommended for heart failure are underused. Indeed, studies have documented that less than 2% of heart failure patients are treated in full compliance with evidence-based treatment guidelines. The process is simply too complex to be adequately practically performed in the human mind.

To assist doctors, software developers have attempted to build machine learning and other types of artificial intelligence tools like those discussed above. However, these tools have generally not been met with widespread success, particularly with medical conditions having properties like those exhibited by heart failure. For example, many types of neural networks exhibit little to no interpretability, meaning that it is difficult or impossible to discern why a particular recommendation is given and verify that the trained model does not suffer from the risk of adversarial input examples or other unexpected, undesirable behavior. Many such models are not provably correct, even if they are right most of the time. The medical community expects greater reliability than many of these types of models can be proven to ensure with commonly used existing software verification techniques. Additional issues arise from the unreliable of data in electronic medical record systems and the lack of human common sense and knowledge in many existing models. Such models typically fail to bring to bear even an approximation of the full range of knowledge possessed by medical professional that can detect and override spurious decisions or recommendations. Other types of artificial intelligence systems, like expert systems, as discussed above, are often too brittle for complex systems with high dimensionality and continuous variables, like the human body. None of this is to suggest that such systems are disclaimed or the any other subject matter described herein is disclaimed, as contemplated embodiments may combine approaches like that disclose below with such techniques to mitigate these and other problems.

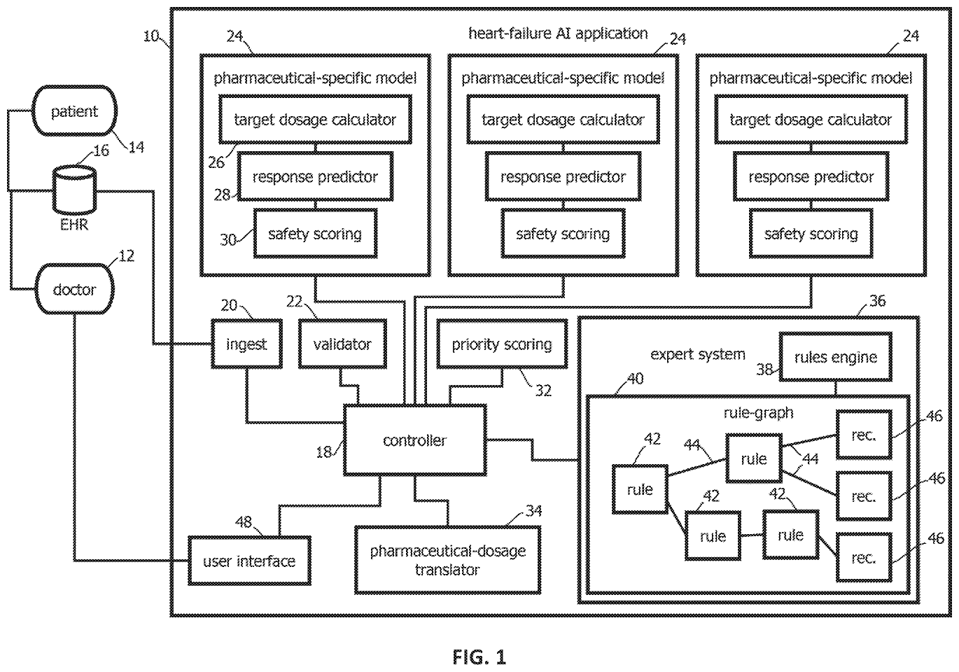

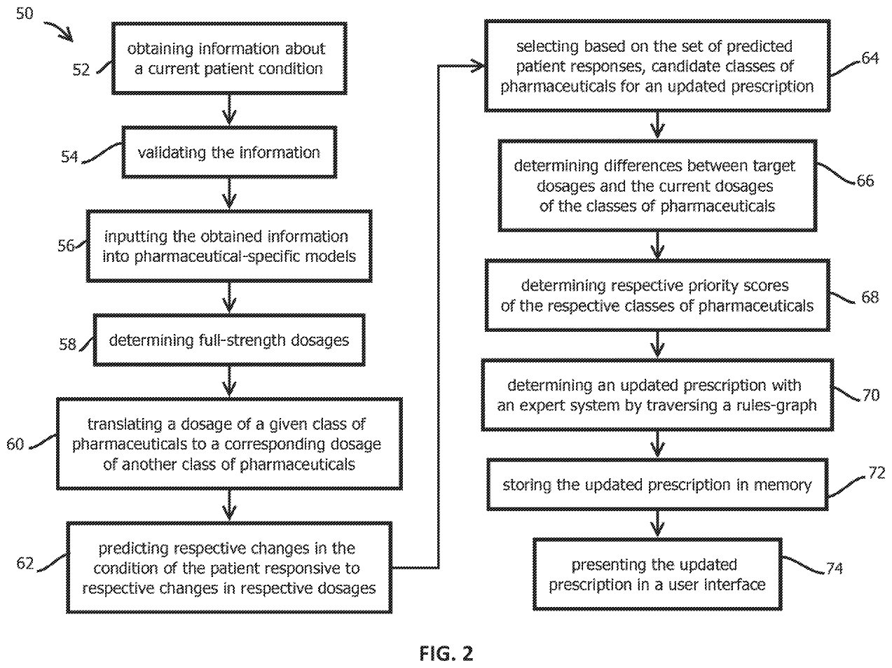

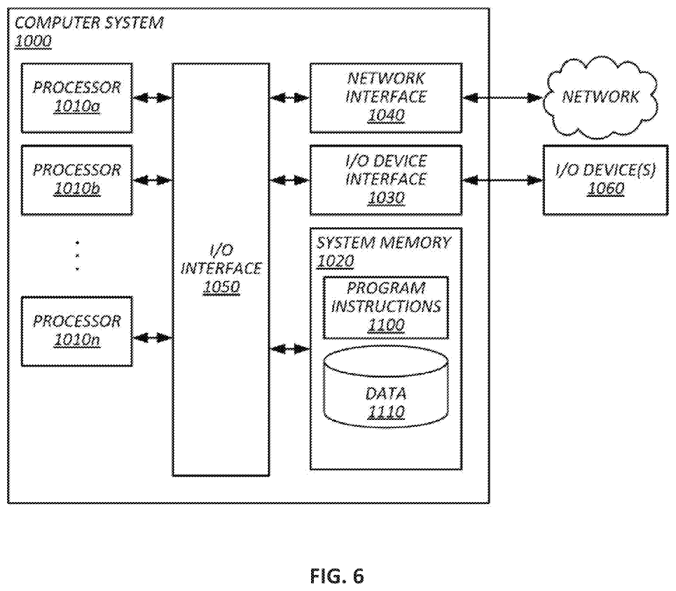

Some embodiments include a software application that assist doctors with treating heart failure in a manner consistent with evidence-based treatment guidelines. Some embodiments implement a software architecture and data model that makes this task tractable in ways that were not feasible with earlier software-based artificial intelligence approaches, and some embodiments implement an iterative, interactive user interface that helps doctors and other medical professionals leverage the recommendations of the present system while understanding how those recommendations are reached and providing inputs by which the doctor can intervene to change various assumptions or recommendations (e.g., to use their judgement to deviate from the guidelines in targeted ways while otherwise according with the guidelines for treatment of heart disease). In some embodiments, the software application may combine a heterogeneous set of models configured to process continuous input and output variables (like lab results and dosages) with an expert system (or other form of symbolic AI) operative to apply relatively complex graphs of Boolean statements or other rules. As a result, the causes and analyses underlying various recommendations may be intelligible, and relatively complex evidence-based treatment guidelines may be encoded in software tools that operate like a “bicycle of the mind” for doctors—i.e., not purporting to replace doctors but serving to make that a doctor's thinking faster and more accurate. Some embodiments implement a logical architecture like that shown in FIG. 1 to execute a process like that shown in FIG. 2 on one or more computer systems like that shown in FIG. 6, as described below. In doing so, some embodiments may traverse rule-graphs like those described below with reference to FIGS. 2 through 5.

As shown in FIG. 1, some embodiments include a heart-failure AI application 10 used by a doctor 12 to treat a patient 14 based on information about the patient 14 stored in an electronic medical record system 16. In some embodiments, the application 10 executes on one or more computing devices like those described below with reference to FIG. 6. In some embodiments, the application 10 is distributed, with different described functionality being implemented on different computing devices, for instance, on a client device in a doctor's office and a remote set of one or more servers in a data center, for instance, by presenting a user interface in a web browser on the client device and serving web content from a remote server system. In another example, the application 10 may be a monolithic application executing on a laptop in a doctor's office. The application may implement supervised learning or unsupervised learning on medical literature and patient records, logic may be hand-coded into the application 10 by software developers working with medical professionals, or some embodiments may apply a hybrid development technique that combines both of these approaches.

In some embodiments, the doctor 12 or other medical professional, such as a nurse, may treat a patient 14 who has experienced heart failure. In some embodiments, the patient may make several visits over several days, weeks, or months, or longer to the doctor 12, and during each visit, the doctor 12 may access the application 10 to execute the process described below with reference to FIG. 2 to update a prescription for the patient in a manner compliant with evidence-based treatment guidelines. In some embodiments, a body of information may accumulate about the patient, and that information may be stored in the electronic medical record system 16 and accessed (for instance, written or read) via various application program interfaces (APIs), for instance, with a Fast Healthcare Interoperability Resources (FHIR) standard-compliant API. In some embodiments, each patient record may be associated with a unique identifier of the patient that distinguishes the patient from other patients, and a variety of fields of information about the patient may be stored in the record, including notes by a physician, lab test results, historical dosages, treatment histories, and the like. In some embodiments, these records may be interrogated by the application 12 and other computer systems via the application program interface, for instance, over a local area network or the Internet. A single doctor 12 and patient 14 are shown, but embodiments are consistent with substantially more users filling these roles, for instance, more than 10 of each, more than 100 of each, or more than 10,000 of each, and while a single EHR system 16 is shown, embodiments may interface with a plurality of different EHR systems, for instance each with different schemas and APIs.

In some embodiments, the application 10 includes an ingest module 20, a validator module 22, a plurality of pharmaceutical-specific models 24, each of which may include a target dosage calculator 26, response predictor 28, and safety scoring module 30. The application 10 may further include a priority scoring module 32, a pharmaceutical-dosage translator 34, and an expert system 36, which may include a rules engine 38 and a rule-graph 40 having a plurality of rules 42 that invoking each other via edges 44 to define logical paths to one or more recommendations 46. The application 10 may further include a user interface 48, such as a visual or audible user interface, like a Web server presenting content in a web browser, a windowing system invoked by a monolithic application and presenting information in an augmented reality head-mounted display worn by a doctor, or a speech-to-text machine learning model operative to interface with a wearable Bluetooth speaker of the doctor. Some embodiments may further include an application program interface by which the described functionality may be invoked by other programs. In some embodiments, the doctor 12 may interact with user interface 48 during data ingest to modify or update data and when viewing results. In some embodiments, the doctor 12 may further use the user interface to change any of the described intermediate results discussed below upon which the final results are based, for example, to change from one class of pharmaceuticals to another or to indicate that a dosage documented in the electronic medical record system 16 is different from that actually taken by the patient. In some embodiments, the operation of these components may be controlled by a controller 18, such as a main program that invokes various subroutines implemented the described functionality and executes a process like that described below with reference to FIG. 2.

In some embodiments, the application 10 includes an ingest module 20. In some embodiments, the doctor 12 may interact with the user interface 48 to specify an identifier of a patient, like a patient name or other unique patient identifier, and the ingest module 20 may send an API request to the electronic medical record system 16 to retrieve information in a corresponding patient file. Examples include patient demographic data, like gender, age, and race. Examples also include historical dosages of various classes of pharmaceuticals and lab results, such as current lab results upon which a current round of changes are to be made to the patient's prescription to change various dosages of various classes of pharmaceuticals.

Examples of lab results include data related to heart disease made by medical device measurements or chemical assays. Examples include impedance cardiograph data based upon variation in impedance measurements during heart beats from an alternating current applied to a set of four or more electrodes placed on the patient adjacent a neck of the patient and a diaphragm of the patient, a measurement of B-type natriuretic peptide (BNP) in blood of the patient, a measurement of N-terminal pro-BNP in blood of the patient, a metabolic panel indicative of an amount of electrolyte imbalance, kidney failure, or liver disease of the patient, a complete blood count (CBC) indicative of anemia, a thyroid tests indicative of an amount of thyroid hormone in blood of the patient, a measurement of Galectin-3 protein in blood of the patient, a measurement of ST2 protein in blood of the patient, and any permutation thereof.

In some embodiments, the retrieved information may be displayed in user interface 48 (which may evolve over time, notwithstanding use of the definite article, for instance “a user interface” and “the user interface” are consistent with reference to a display screen that evolves over time, with the display screen changing between the references, that is “the user interface” can present different information than the antecedent “a user interface” consistent with such evolution). In some embodiments, validator 22 may compare the retrieved information (or changes relative to previous values) to various sets of thresholds for each field of information and flag values that exceed a maximum threshold or are below a minimum threshold to identify potentially anomalous, incorrectly entered values. In some embodiments, the formatting of such values or other visual indications may be provided in a display screen in response to determining that such thresholds are satisfied. In some embodiments, the user interface may include inputs by which a doctor may manually change various values or supply new values upon which subsequent computations are based.

In some embodiments, the validated information may identify a plurality of classes of pharmaceuticals taken or to be taken by the patient and corresponding doses. Based on this information, some embodiments of the controller 18 may invoke models 24 corresponding to those classes of pharmaceuticals. In some embodiments, the subset may include a plurality of such models 24, such as two, three, four, or more, and the subset may be selected from a plurality of models 24, like four more, six or more, or ten or more. In some embodiments, each model 24 may correspond to a different class of pharmaceuticals, e.g., corresponding to a different active ingredient, or corresponding to different brands. Examples of relevant classes of pharmaceuticals that may each have their own distinct model 24 include an angiotensin-converting enzyme (ACE) inhibitor model, an angiotensin receptor blocker model, a beta-blocker model, a mineralocorticoid-receptor antagonist (MRA) model, and a renin-angiotensin-system (RAS) blocker model. In some embodiments, the models 24 may be invoked by passing or passing a pointer to inputs from the validated information upon which the models operate.

In some embodiments, the models 24 are configured to operate upon continuous variables, like lab results, age, weight, height, and dosages as well as nominal values, like gender and race. In some embodiments, the models 24 receive these inputs and produce an output indicative of a candidate recommended change in dosage of the corresponding class of pharmaceuticals, for instance, to decrease, increase, or leave unchanged a current dosage. In some embodiments, the output is a safety score like that described below, for instance, ranging from −100 to 100, with a value of zero indicating that the safest course would be to leave the current dosage unchanged, a score of positive 100 indicating the safest course is to increase by a relatively large amount, a positive score of 50 indicating the safest course is to increase by a smaller amount, a negative score of −50 indicating the safest course is to decrease by the smaller amount, and a negative score of −100 indicating the safest course is to decrease the dosage by relatively large amount or eliminate the dosage. Or signs may be reversed, and ranges may be implemented on different scales to express similar logic. In some embodiments, the output is an ordinal value ranging from decrease, leave unchanged, to increase. In some embodiments, a similar approach may be applied with greater or less granularity. These outputs, along with an associated priority score for module 32 may be supplied as inputs to the expert system 36, as described below.

In some embodiments, the models 24 based these outputs on various intermediate computations. For example, some embodiments may include the target dosage calculator 26 that computes a target dosage for the patient given the attributes of the patient. The target dosage may be a full dosage to be achieved after potentially several rounds of incremental adjustments upward towards the target dosage. The target dosage may be specified based upon treatment guidelines to neutralize or mitigate various causes of adverse effects from heart failure. In some embodiments, the target dosages may be compared to a current dosage and a difference may be computed. In some embodiments, the differences may be normalized, for instance, expressing the current dosage as a percentage of the target dosage.

In some embodiments, the models 24 may include a response predictor 28. In some embodiments, the response predictor may predict the effect of increasing or decreasing the current dosage by an increment specified in memory, for instance, a default value subject to modification by the doctor 12 via the user interface 48. In some embodiments, effects may be predicted with respect to a plurality of dimensions of patient health. For example, RAS and MRA may affect total peripheral resistance (TPRI), and beta-blockers may affect heart rate (HR). In some embodiments, effects may be predicted with other approaches, like a dynamic Bayesian network, decision tree (or random forest thereof), or neural network trained on historical patient data.

In some embodiments, each of these predicted effects may be input into the safety scoring module 30, which may compute an aggregate safety score based upon each of the effects. In some embodiments, the aggregate safety score is a cardinal value like that described above or one of the ordinal outputs like those described above, which in some cases may be based upon a cardinal value and various thresholds. For example, some embodiments may compare such a cardinal value to an upper threshold and a lower threshold and designate cardinal values therebetween as corresponding to no change, designate cardinal values above the upper threshold as corresponding to an increase in dosage, and designate cardinal values below the lower threshold as designating a decrease in dosage. In some embodiments, such cardinal values may be computed as a weighted average of the predicted effects, with the predicted effects being normalized and scored as cardinal values themselves. Some embodiments may combine various predicted effects in an ensemble model that aggregates these signals into a single output or set of outputs, e.g., with the various types of machine learning models described above. In some cases, safety scores or related values may be adjusted based on an amount of change (e.g., rate of change in or delta between measurements) in a patient's glomerular filtration rate (GFR). Some embodiments may obtain a value indicative of such a change, compare that value to a threshold, and set a flag in program state in response to determining the threshold is exceeded. This flag may cause a user interface like those described below to be adjusted to indicate the change and its potential consequences.

In some examples, the safety score for the RAS class of drugs may weigh the relative distance from a target K+ (potassium) value and then may yield exponentially increasing negative values as it decreases outside the normally accepted lower range limit. Similarly, the safety score may be simultaneously affected by low and lowering eGFR (estimated glomerular filtration rate) lab test results in a negative sense. The safety score may be one or more values based on the combination and interaction effects of the various inputs in a non-linear response. A K+ value within the normal acceptable range and higher and stable eGFR value may yield a score indicating it is acceptable to start or increase the dosage of the RAS and MRA drug classes. Marginal or near acceptable range limits of K+, eGFR and ΔeGFR (change in eGFR) may yield a score indicating it is advisable to maintain the current dosage or to not initiate RAS or MRA therapy at a current visit. A significantly large negative score, influenced by low K+, low eGFR or decreasing ΔeGFR (or some combination), may indicate that the RAS therapy should be reduced or stopped. For example, the safety score for the RAS class of drugs may be based on current potassium (K+) and kidney function eGFR/creatinine lab test results, as well as the percentage change from a recently prior eGFR lab test with the following equation: RAS safety score=−11.7+4.5*K++0.2*eGFR-0.1*ΔeGFR+0.0125*K+*ΔeGFR-0.6*K+2−0.0015*eGFR2. This equation may produce values relative to a scale of −5 to +5. Thus, if K+=4, current eGFR=50, and prior eGFR=60 (ΔeGFR=16.7), then the RAS safety score is about 2. This resulting safety score, in the context of the relative scale, may indicate it is acceptable to consider starting or increasing the current dosage of the RAS class drug if not already on max tolerated dose. If K+=5.5, eGFR=40 and prior eGFR=60 (ΔeGFR=33), then the RAS safety score is less than −0.50, which may indicate that the current dosage should be reduced or delay starting RAS therapy if not already taking.

In some examples, a beta-blocker safety score may be based on the patient's current blood pressure (SBP/DBP) and heart rate. For example, the pulse pressure (pp=SBP-DBP, systolic—diastolic pressure) may be utilized, and the Beta safety score may equal or be based on: −80.6+1.4* SBP−0.007*pp+1. 04*HR+0.0003* SBP*HR−0.000075*pp*HR−0.0125* SBP2−0.0134*HR2+0.00004*SBP3+0.00005*HR3. This equation is also relative to a scale of −5 to +5. Thus, if BP is 100/50 (pp=50) and HR is 85, then the beta safety score is about 0.6. Since this score is relatively close to 0, the recommendation may be to not start beta-blocker therapy or maintain the current dosage is already taking a beta-blocker.

In some embodiments, effects and safety scoring are proportional to various inputs, or in some cases effects and safety scoring are multivariate outputs that are nonlinearly related to inputs. In some embodiments one or more of these values may be based upon a linear regression, a neural network trained on historical patient data (like a recurrent neural network, such as a long-short term memory model or an autoencoder with attention, such as a transformer model), hardcoded equations, a decision tree trained on historical patient data, a dynamic Bayesian network (e.g., a hidden Markov model) trained on historical patient data, or a reinforcement learning model trained on historical patient data. In some embodiments, each of the models 24 may have a similar architecture, but equations, parameters, and thresholds thereof may differ. Outputs of each may be expressed, though, in a similar schema, for instance as a safety score.

The safety scores or related values may be advanced to the priority scoring module 32, in some cases along with differences between target and current dosages. In some embodiments, the priority scoring module 32 may compute a priority for each class of pharmaceuticals to increase that class of pharmaceuticals, for instance, based upon the urgency of the medical effects to be mitigated. In some cases, each model 24 has a different priority scoring module 32, which is not to suggest that other features are limited to the logical architecture shown in FIG. 1. In some embodiments, outputs for each of the different classes of pharmaceuticals may produce different priority scores for each class, and in some cases, the priority scores may be on a shared range to facilitate comparison between priorities of different classes of pharmaceuticals during downstream operations. The priority scoring may involve two inputs (or more or less). The first input may be the relative distance from target values for TPRI and HR. The RAS and MRA drug classes may be related to TPRI and the beta-blockers may be related to HR. Since TPRI and HR are on dramatically different scales, in some embodiments, they may be rationalized to determine greatest relative distance. Re-scaling equations of those current values to a common scale may be utilized to achieve a relative distance from target. The greater of those two relative distances may be assigned the higher priority. The second input for priority may be the currently prescribed dosage (assuming the patient is adhering to the drug regimen). All three drug classes of the triple therapy evidence-based standard may be balanced against each other and against the max target dosage. The current dosages may be converted to a percentage of the max target dose. The priority equations may then score the classes such that the balances are intended to promote a near equal progression to the target max dose of each class. The two priority influencing scores, along with an overriding safety score, may determine the recommendation of drug therapy action at a current visit. The recommendation may be limited to no more than two drug therapy changes at any one visit. Often the recommendation may suggest only one change or none.

In some embodiments, a physician may input via the user interface 48 a request to substitute one class of pharmaceuticals for another, and some embodiments may invoke the pharmaceutical-dosage translator 34 to translate dosages therebetween. In some embodiments, the translator 34 may maintain an index (e.g., a table, like a matrix) by which a dosage in one class of pharmaceuticals may be translated into that of another. In some embodiments, a current dosage may be translated with the translator 34 and the translated dosage may be input as a current dosage to the model 24 corresponding to the new class of pharmaceuticals to which the patient is to be potentially transitioned. Such features are expected to facilitate interactive “what-if” analysis by physicians as the explore consequences of possible treatment approaches.

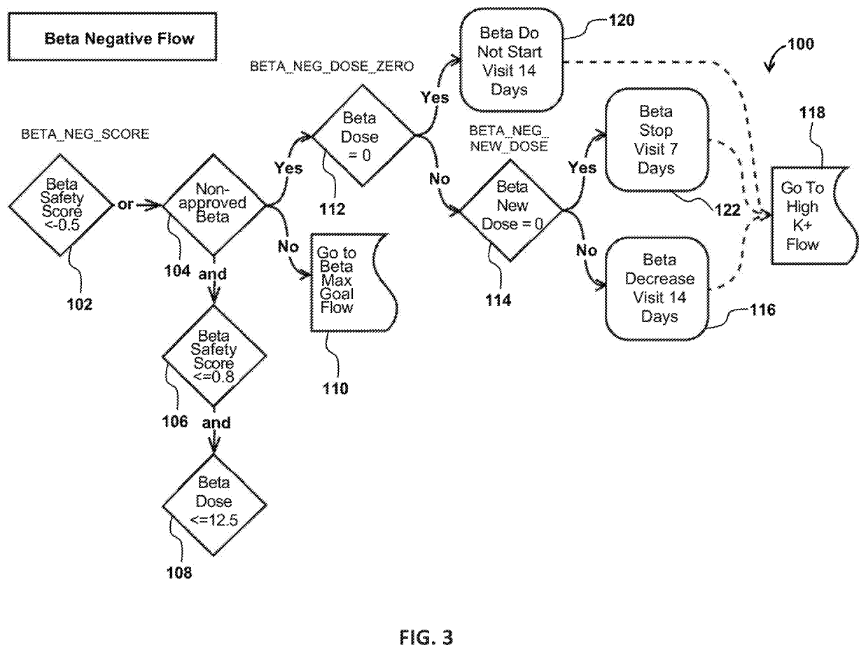

In some embodiments, the safety scores (or values based thereon) and priority scores (which may also be expressed as cardinal, nominal, or ordinal values) for each class of pharmaceuticals may be advanced by the controller 18 to the expert system 36. In some embodiments, the system 36 includes the rules engine 38 and the rule-graph 40. In some embodiments, the rules engine 38 may be configured to traverse the rule-graph 40 and evaluate Boolean statements (in some cases with compound Boolean operators by which more complex statements are composed of simpler statements) at each node to determine which edges 44 to traverse. Or some embodiments may include other types of statements, such as statements with more than binary outputs. Example rules may specify that not more than a threshold number of classes of pharmaceuticals are to be taken at a given time and not more than a threshold number of classes of pharmaceuticals are be to changed upward in dosage in a given time. Other examples of rules may include if-then statements that turn on a threshold for various lab results, priority scores, safety scores, or combination thereof. In some embodiments, depending upon this logic, the safety score may override other considerations, and when the safety score is in an acceptable range, other criteria may be outcome determinative. As a result, the rules may dynamically change recommendations based on what is safe and what is being prioritized in ways that implement relatively rich bodies of logic. In some embodiments, the rule-graph 40 may be embodied by an abstract syntax tree formed by parsing a domain specific programming language by which rules are encoded in source-code form. Examples of various portions of a rule-graph 40 are described below with reference to FIGS. 3 through 5.

In some embodiments, rules may take variables, like outputs of other rules, outputs of the model 24, or patient data that is ingested and validated. In some embodiments, the rule-graph 40 includes a relatively large number of rules, like more than 100 rules, more than 200 rules, more than 300 rules, or more than a thousand rules. In some embodiments, rules may specify conditional branches, and criteria by which those conditional branches are selected. In some embodiments, the conditional branches may correspond to edges 44 by which other rules are invoked or variables are changed. Some branches may correspond to multiple edges.

In some embodiments, some of the rules may terminate in recommendations 46. Example recommendations may include increasing, decreasing, or leaving unchanged a given class of pharmaceuticals and various other interventions or instructions for a physician 12, like additional lab tests or other things to monitor. For example, one recommendation may be to refer the patient to an emergency room for intravenous fluid administration. Another recommendation may be to increase fluid intake. Another recommendation may be to decrease a particular class of pharmaceutical's dosage by a designated amount. In some cases, such logic may trigger multiple recommendations or only one.

FIG. 2 illustrates an example of a process 50 that may be executed by the application 10 described above or other software applications. In some embodiments, the operations may be encoded on a tangible, non-transitory, computer readable medium, like memory, for instance as program code, such that when the instructions are executed by one or more processors, the described operations may be implemented. The described operations may be executed in a different order, some operations may be repeated, some operations may be executed concurrently, operations may be executed sequentially, and additional operations may be added, none of which is to suggest that other described features are not also amenable to variation.

In some embodiments, the process 50 includes obtaining information about a current patient condition, as indicated by block 52. In some embodiments, this may be performed by the above-described ingest module 20 and the user interface 48. The obtained information may include, for instance, current dosages and lab results since a previous session with the application 10 for a given patient, along with demographic attributes of the patient.

Embodiments may include validating the information, as indicated by block 54, for instance with the above-described validator 22 and user interface 48. In some cases, a doctor may change values retrieved from the EHR 16 and potentially anomalous values out of expected ranges may be detected and visually designated in a display differently from values determined to not be anomalous, e.g., with a different font, color, alarm headers, etc.

Embodiments may further include inputting the obtained information into pharmaceutical-specific models, as indicated by block 56, or other types of intervention-specific models (e.g., drugs, exercise and diet changes, treatments, use of medical devices, etc.). This may include selecting among the above-described models 24 and in some cases translating a current dosage for one class of pharmaceuticals into another debt class of pharmaceuticals with the translator 34 described above, before selecting a model 24 for the new class of pharmaceuticals.

Some embodiments include determining full-strength dosages for the patient based on the patient information, as indicated by block 58, for instance with the target dosage calculated as described above with reference to element number 26 and FIG. 1. This may be done for each of a plurality of different classes of pharmaceuticals having corresponding models 24 that were invoked.

Some embodiments may translate a dosage of a given class of pharmaceuticals to a corresponding dosage of another class of pharmaceuticals, as indicated by block 60, for instance, with the above-described pharmaceutical-dosage translator 34.

Some embodiments may include predicting respective changes in the condition of the patient responsive to the respective changes in respective dosages, as indicated by block 62. Some embodiments may account for interactions between different pharmaceuticals in the models and provide conditional safety scores, for instance, a set of safety scores corresponding to different scenarios in which different pairs of drugs are adjusted. As noted, in some cases this may include predicting changes in a plurality of different dimensions of patient health and aggregating those changes in an aggregate safety score.

Some embodiments may include selecting based on the predicted patient responses, candidate classes of pharmaceuticals for an updated prescription, as indicated by block 64. In some embodiments, this may include comparing safety scores to thresholds corresponding to decreasing, leaving unchanged, and increasing the class of pharmaceuticals as a candidate adjustment.

Some embodiments include determining differences between target dosages and current dosages of the classes of pharmaceuticals, as indicated by block 66. In some embodiments, this may be done before predicting respective changes in block 62, which is not to suggest that the sequence is otherwise limited to that depicted in FIG. 2. In some embodiments, these differences may be normalized on a scale shared by the different classes of pharmaceuticals, like between zero and a hundred percent of target.

Some embodiments may determine respective priority scores of the respective classes of pharmaceuticals, as indicated by block 68. In some embodiments, these priority scores may be based on the difference calculated in block 66, with larger differences corresponding to higher priorities in some cases. In some cases, this correspondence may be proportional to such differences, or it may vary non-linearly, e.g., exponentially with the difference or as a log of the difference.

In some embodiments, the process 50 may include determining updated prescription with an expert system by traversing a rules graph, as indicated by block 70. In some embodiments, this may be done by the above-described expert system 36 traversing rule-graphs like those described below with reference to FIGS. 3 through 5. In some embodiments, the updated prescription may include a plurality of recommendations like those described above and may include adjusting dosages relative to a current dosage of one or more classes of pharmaceuticals as part of an updated prescription.

Some embodiments may store the updated prescription (or other set of interventions) in memory, as indicated by block 72, and present the updated prescription in a user interface, as indicated in block 74. The presentation may take a variety of forms, examples which are described above, and may include graphical elements that adjust the visual weight of information based upon its importance. For example, a heavier font, larger font, different font, certain colors like the color red, or position on the screen may be adjusted responsive to determining that a particular recommendation is particularly important to increase its visual weight relative to less important information, for instance, responsive to an outcome of traversing the rule-graph by evaluating Boolean statements therein. In some embodiments, some of the recommendations 46 described above may correspond to formatting adjustments of other recommendations to adjust the visual weight with which they are presented.

FIG. 3 is a flowchart illustrating an example of a portion of a rule-graph 100 pertaining to a negative state with respect to beta. Some embodiments include determining whether the betas safety score is less than −0.5 or some other threshold value or the patient is on a non-approved beta, as indicated by block 104. Some embodiments further include determining whether the beta safety score is less than 0.8, as indicated by block 106, with an “and” operator connecting these criteria and determining whether the beta dose is less than or equal to 12.5% (e.g., of a target dosage), as indicated by block 108. Some embodiments include conditional branches that direct the rules engine to go to a different portion of the rules graph, as indicated by linking icon 110 in response to a negative determination of the preceding logical predicates, and some embodiments in the alternative proceed to block 112 to determine whether the beta dose is equal to zero. Responsive to an affirmative determination, some embodiments may trigger a recommendation 120 that beta dosage should not start and the patient should visit the doctor again in 14 days. Alternatively, some embodiments may proceed to determine whether the new candidate dosage for beta is equal to zero, as indicated by block 114. In response to an affirmative determination, embodiments may trigger a recommendation 122 that the beta dosage should be stopped and the patient should visit the doctor again in seven days. Alternatively, embodiments may proceed to block 116 and decrease beta dosage, for instance, by a predetermined amount by which increments are to be made and instruct the patient to visit in 14 days, as indicated by a recommendation 116. Each of these recommendations 120, 122, and 116 may proceed via a link to another portion of the rule-graph, as indicated by block 118.

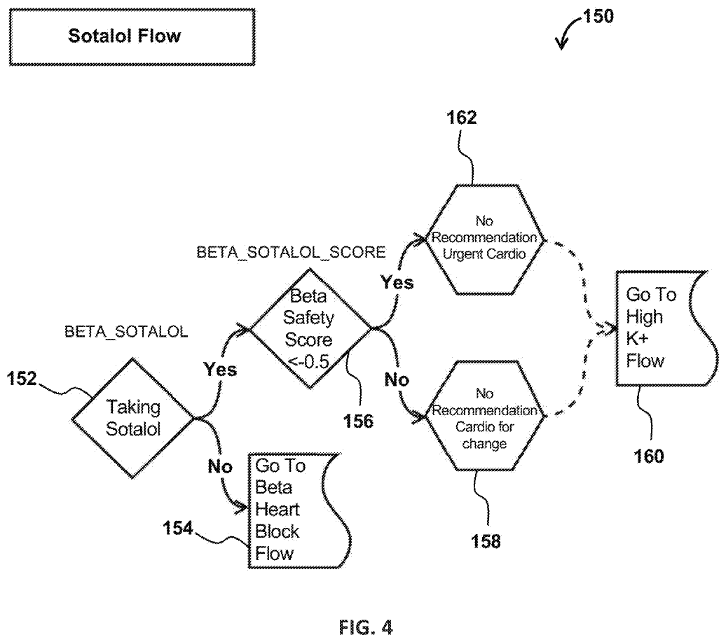

Some embodiments may include a portion of a rule-graph 150 illustrated in FIG. 4 related to patient use of sotalol. Embodiments may include determining whether the patient is taking sotalol, as negated by block 152. Upon determining they are not, embodiments may proceed to link 154 to another portion of the rules graph. Upon determining that they are, embodiments may proceed to block 156 to determine whether the beta safety scores less is than −0.5. Upon determining that it is not, embodiments may proceed to make a no recommendation of cardio for a change, as indicated of the light block 158. Alternatively, embodiments may proceed to block 162 and make a no recommendation and early and urgent cardio, as indicated by block 162. In either case, embodiments may proceed to another portion of the rules graph indicated by link 160.

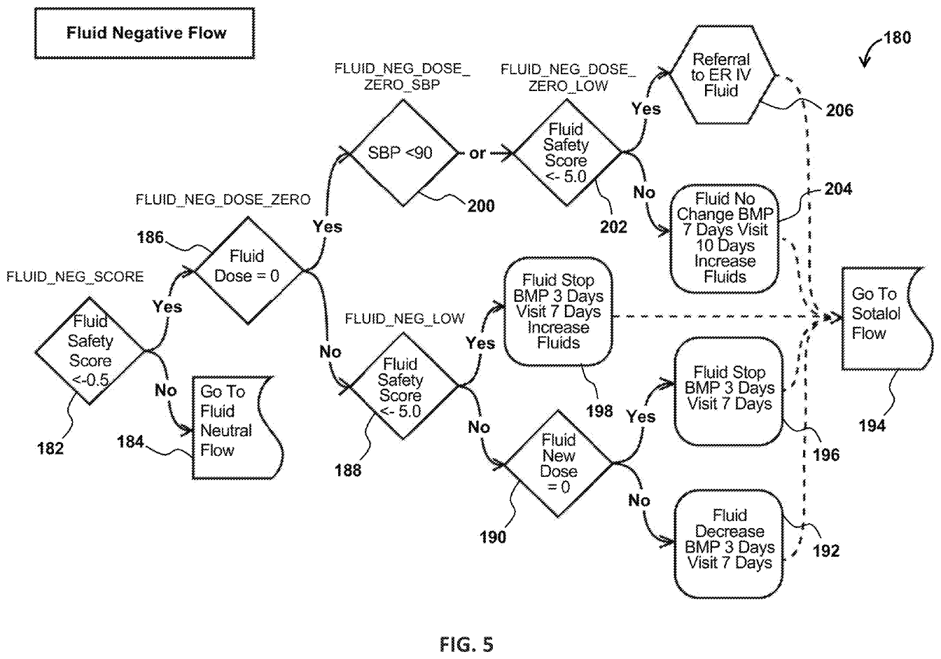

FIG. 5 illustrates another portion of a rules graph 180 related to fluid levels of the patient. Embodiments may determine whether the fluid safety score is less than −0.5, as indicated by block 182. Upon determining that it is not, embodiments may proceed to a link 184 to another portion of the rule-graph related to fluid levels being neutral. Alternatively, embodiments may proceed to determine whether the fluid dosage is equal to zero, as indicated by block 186. Upon determining that it is not, embodiments may determine whether a fluid safety score is less than −5.0, as indicated by block 188. In response to a negative determination, embodiments may proceed to determine whether a fluid new dosage is equal to zero, as indicate by block 190. Upon determining that it is not, embodiments may recommend a fluid decrease, a basic metabolic panel (BMP) for or in three days, and a visit to the doctor in seven days, as indicated by recommendation 192. Alternatively, embodiments may recommend a fluid dosage stop, a BMP for or in three days, and a visit to the doctor in seven days, as indicated by block 196. In response to an affirmative determination at block 188, embodiments may recommend a fluid stop, a BMP in or for three days, a visit to the doctor in seven days, and an increase in fluids, as indicated by block 198. In response to an affirmative determination in block 186, embodiments may proceed to determine whether either systolic blood pressure (SBP) is less than 90 or a fluid safety score is less than −5.0, as indicated by blocks 202 and 200. In response to a negative determination, some embodiments may recommend no changed fluids, a BMP in or for seven days, a visit to the doctor in 14 days, and an increase in fluids, as indicated by block 204. In the alternative, embodiments may refer the patient to an emergency room for intravenous fluid administration, as indicated by block 206. Concluding each of these recommendations, embodiments may proceed to another portion of the rule-graph corresponding to FIG. 4, as indicated by link 194. Based on these rules, some embodiments may determine changes to a current intervention state (e.g., drugs, dosages thereof, and other medical interventions, like IV fluids) and produce an updated recommended intervention set (e.g., a prescription).

FIG. 6 is a diagram that illustrates an exemplary computing system 1000 in accordance with embodiments of the present technique. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:
1. A tangible, non-transitory, machine-readable medium storing instructions that when executed by one or more processors effectuate operations comprising: obtaining, with an artificial intelligence (AI) application executed by a computer system, information about a current patient condition, wherein: the information relates to a patient who has undergone heart failure, at least some of the information is obtained from an electronic medical records system via an application program interface of the electronic medical records system, at least some of the information is validated or entered into the AI application by a medical professional after obtaining information from the electronic medical records system, and the AI application comprises: a plurality of pharmaceutical-specific models each corresponding to a different class of pharmaceuticals to treat heart failure and each configured to predict a response of the patient to a change in dosage of the corresponding class of pharmaceuticals given the current patient condition, and an expert system comprising a plurality of rules in a rule-graph, the rule-graph being traversable to determine an updated prescription based on the patient condition and the predicted patient responses; inputting, with the computer system, at least some of the obtained information about the patient condition into the respective pharmaceutical-specific models and, in response: predicting respective changes in the condition of the patient responsive to respective changes in respective dosages of the respective classes of pharmaceuticals to obtain a set of predicted patient responses for the different classes of pharmaceutical, selecting based on the set of predicted patient responses, candidate classes of pharmaceuticals for an updated prescription, determining differences between target dosages and the current dosages of the classes of pharmaceuticals, and based on the differences, determining respective priority scores of the respective classes of pharmaceuticals; inputting, with the computer system, into the expert system of the AI application, the candidate classes of pharmaceuticals and their respective priority scores and, in response, determining an updated prescription; and storing, with the computer system, the updated prescription in memory.
2. The medium of embodiment 1, wherein the pharmaceutical-specific models comprise at least two of the following: an angiotensin-converting enzyme (ACE) inhibitor model, an angiotensin receptor blocker model, a beta-blocker model, a mineralocorticoid-receptor antagonist (MRA) model, or a renin-angiotensin-system (RAS) blocker model.
3. The medium of embodiment 1, wherein the pharmaceutical-specific models comprise each of the following: an angiotensin-converting enzyme (ACE) inhibitor model, an angiotensin receptor blocker model, a beta-blocker model, a mineralocorticoid-receptor antagonist (MRA) model, and a renin-angiotensin-system (RAS) blocker model.
4. The medium of any one of embodiments 1-3, wherein: at least some of the pharmaceutical-specific models comprise: a sub-model configured to determine a full-strength dosage of the respective class of pharmaceuticals for the patient based on the information about the current patient condition, an input configured to receive a current dosage of the respective class of pharmaceuticals for the patient from the information about the current patient condition, and a plurality of thresholds indicative of whether the current dosage is to be determined to be safe to increase, decrease, or leave unchanged for the patient; and the at least some of the pharmaceutical-specific models are configured to determine, based on the information about the current patient condition, a respective value indicative of whether the current dosage is determined to be safe and compare that value to the plurality of thresholds to determine whether to recommend a medical professional increase, decrease, or leave unchanged the current dosage.
5. The medium of any one of embodiments 1-4, wherein: the AI application comprises a translator configured to translate a dosage of a given class of pharmaceuticals to a corresponding dosage of another class of pharmaceuticals; and a model specific to the another class of pharmaceuticals is configured to use the translated dosage to predict a patient response to changes in the another class of pharmaceuticals based on a current dosage in the given class of pharmaceuticals.
6. The medium of embodiment 4, wherein the AI application is configured to determine that more than a threshold number of candidate classes of pharmaceuticals are determined to be candidates to be increased and, in response, select a subset of the candidate classes of pharmaceuticals to be increased based on respective determined priority scores of the subset of the candidate classes of pharmaceuticals.
7. The medium of any one of embodiments 1-6, wherein the AI application is configured to: determine a value indicative of a difference in heart rate between a target heart rate of the patient and a patient heart rate indicated in the information about the current patient condition; and adjust or otherwise determine at least some of the priority scores based on the value indicative of the difference in heart rate.
8. The medium of any one of embodiments 1-7, wherein the AI application is configured to: determine a value indicative of a difference in thrombolytic predictive instrument (TPI) measurements between a target TPI measurement of the patient and a patient TPI measurement in the information about the current patient condition; and adjust or otherwise determine at least some priority scores based on the value indicative of the difference in TPI measurements.
9. The medium of any one of embodiments 1-8, wherein the expert system comprises: a plurality of rules encoded as Boolean statements corresponding to nodes and edges of the rule-graph; and a rules engine configured to: traverse the rule-graph by evaluating the Boolean statements based the candidate classes of pharmaceuticals and their respective determined priority scores, and output an updated prescription based on the evaluating.
10. The medium of any one of embodiments 1-9, wherein the expert system comprises a plurality of rules including at least two of the following: a rule responsive to whether fluid flow in the patient is negative; a rule responsive to whether fluid flow in the patient is neutral; a rule responsive to whether the patient is taking an antiarrhythmic agent; a rule responsive to whether to whether the patient has experienced atrial fibrillation; a rule responsive to an whether a threshold amount of dosages of different classes of pharmaceuticals are candidates to be changed concurrently; or a rule that adjusts the threshold amount of dosages of different classes of pharmaceuticals based on a blood sugar level of the patient in the information about the current patient condition.
11. The medium of any one of embodiments 1-10, wherein the expert system comprises a plurality of rules including each of the following: a rule responsive to whether fluid flow in the patient is negative; a rule responsive to whether fluid flow in the patient is neutral; a rule responsive to whether the patient is taking an antiarrhythmic agent; a rule responsive to whether to whether the patient has experienced atrial fibrillation; a rule responsive to an whether a threshold amount of dosages of different classes of pharmaceuticals are candidates to be changed concurrently; and a rule that adjusts the threshold amount of dosages of different classes of pharmaceuticals based on a blood sugar level of the patient in the information about the current patient condition.
12. The medium of any one of embodiments 1-11, wherein the expert system comprises a plurality of rules including each of the following: a rule that takes a variable; a rule that modifies a value of a variable in another rule conditional upon inputs; a rule that outputs a recommendation conditional upon inputs; and a rule that that outputs a prohibition conditional upon inputs.
13. The medium of any one of embodiments 1-12, wherein: the expert system comprises more than 250 rules; the prescription is updated more than 5 times for a given patient; the number of prescription-specific models is greater than or equal to 4; and the AI model is responsive to more than 10 different lab test measurements and demographic attributes.
14. The medium of any one of embodiments 1-13, wherein the AI application is configured to present a recommended updated prescription in a user interface based on output of the expert system.
15. The medium of any one of embodiments 1-14, wherein the information about the patient comprises: impedance cardiograph data based upon variation in impedance measurements during heart beats from an alternating current applied to a set of four or more electrodes placed on the patient adjacent a neck of the patient and a diaphragm of the patient; age and gender of the patient; a value indicative of use of a loop diuretic with the patient; and values indicative of results of at least three of the following tests of the patient: a measurement of B-type natriuretic peptide (BNP) in blood of the patient, a measurement of N-terminal pro-BNP in blood of the patient, a metabolic panel indicative of an amount of electrolyte imbalance, kidney failure, or liver disease of the patient, a complete blood count (CBC) indicative of anemia, a thyroid tests indicative of an amount of thyroid hormone in blood of the patient, a measurement of Galectin-3 protein in blood of the patient, or a measurement of ST2 protein in blood of the patient.
16. A method comprising: the operations of any one of embodiments 1-15.
20. A system, comprising: one or more processors; and memory storing instructions that when executed by the processors cause the processors to effectuate operations comprising: the operations of any one of embodiments 1-15.

What is claimed is:

1. A tangible, non-transitory, machine-readable medium storing instructions that when executed by one or more processors effectuate operations comprising:
obtaining, with an artificial intelligence (AI) application executed by a computer system, information about a current patient condition, wherein:
the information relates to a patient who has undergone heart failure,
at least some of the information is obtained from an electronic medical records system via an application program interface of the electronic medical records system,
at least some of the information is validated or entered into the AI application by a medical professional after obtaining information from the electronic medical records system, and
the AI application comprises:
a plurality of pharmaceutical-specific models each corresponding to a different class of pharmaceuticals to treat heart failure and each configured to predict a response of the patient to a change in dosage of the corresponding class of pharmaceuticals given the current patient condition, and
an expert system comprising a plurality of rules in a rule-graph, the rule graph being traversable to determine an updated prescription based on the patient condition and the predicted patient responses;
inputting, with the computer system, at least some of the obtained information about the patient condition into the respective pharmaceutical-specific models and, in response:
predicting respective changes in the condition of the patient responsive to respective changes in respective dosages of the respective classes of pharmaceuticals to obtain a set of predicted patient responses for the different classes of pharmaceutical,
selecting based on the set of predicted patient responses, candidate classes of
pharmaceuticals for an updated prescription, determining differences between target dosages and the current dosages of the classes of pharmaceuticals, and
based on the differences, determining respective priority scores of the respective classes of pharmaceuticals;
inputting, with the computer system, into the expert system of the AI application, the candidate classes of pharmaceuticals and their respective priority scores and, in response, determining an updated prescription; and
storing, with the computer system, the updated prescription in memory.

2. The medium of claim 1, wherein the pharmaceutical-specific models comprise at least two of the following:
an angiotensin-converting enzyme (ACE) inhibitor model,
an angiotensin receptor blocker model,
a beta-blocker model,
a mineralocorticoid-receptor antagonist (MRA) model, or
a renin-angiotensin-system (RAS) blocker model.

3. The medium of claim 1, wherein the pharmaceutical-specific models comprise each of the following:
an angiotensin-converting enzyme (ACE) inhibitor model,
an angiotensin receptor blocker model,
a beta-blocker model,
a mineralocorticoid-receptor antagonist (MRA) model, and
a renin-angiotensin-system (RAS) blocker model.

4. The medium of claim 1, wherein:
at least some of the pharmaceutical-specific models comprise:
a sub-model configured to determine a full-strength dosage of the respective class of pharmaceuticals for the patient based on the information about the current patient condition,
an input configured to receive a current dosage of the respective class of pharmaceuticals for the patient from the information about the current patient condition, and
a plurality of thresholds indicative of whether the current dosage is to be determined to be safe to increase, decrease, or leave unchanged for the patient; and
the at least some of the pharmaceutical-specific models are configured to determine, based on the information about the current patient condition, a respective value indicative of whether the current dosage is determined to be safe and compare that value to the plurality of thresholds to determine whether to recommend a medical professional increase, decrease, or leave unchanged the current dosage.

5. The medium of claim 1, wherein:
the AI application comprises a translator configured to translate a dosage of a given class of pharmaceuticals to a corresponding dosage of another class of pharmaceuticals; and
a model specific to the another class of pharmaceuticals is configured to use the translated dosage to predict a patient response to changes in the another class of pharmaceuticals based on a current dosage in the given class of pharmaceuticals.

6. The medium of claim 4, wherein the AI application is configured to determine that more than a threshold number of candidate classes of pharmaceuticals are determined to be candidates to be increased and, in response, select a subset of the candidate classes of pharmaceuticals to be increased based on respective determined priority scores of the subset of the candidate classes of pharmaceuticals.

7. The medium of claim 1, wherein the AI application is configured to:
determine a value indicative of a difference in heart rate between a target heart rate of the patient and a patient heart rate indicated in the information about the current patient condition; and
adjust or otherwise determine at least some of the priority scores based on the value indicative of the difference in heart rate.

8. The medium of claim 1, wherein the AI application is configured to:
determine a value indicative of a difference in thrombolytic predictive instrument (TPI) measurements between a target TPI measurement of the patient and a patient TPI measurement in the information about the current patient condition; and
adjust or otherwise determine at least some priority scores based on the value indicative of the difference in TPI measurements.

9. The medium of claim 1, wherein at least some of the pharmaceutical-specific models comprise:
means for determining whether the respective class of pharmaceuticals is suitable for the patient; and
means for determining a priority of the respective class of pharmaceuticals.

10. The medium of claim 1, wherein the expert system comprises:
a plurality of rules encoded as Boolean statements corresponding to nodes and edges of the rule-graph; and
a rules engine configured to:
traverse the rule-graph by evaluating the Boolean statements based the candidate classes of pharmaceuticals and their respective determined priority scores, and
output an updated prescription based on the evaluating.

11. The medium of claim 1, wherein the expert system comprises a plurality of rules including at least two of the following:
a rule responsive to whether fluid flow in the patient is negative;
a rule responsive to whether fluid flow in the patient is neutral;
a rule responsive to whether the patient is taking an antiarrhythmic agent;
a rule responsive to whether to whether the patient has experienced atrial fibrillation;
a rule responsive to an whether a threshold amount of dosages of different classes of pharmaceuticals are candidates to be changed concurrently; or
a rule that adjusts the threshold amount of dosages of different classes of pharmaceuticals based on a blood sugar level of the patient in the information about the current patient condition.

12. The medium of claim 1, wherein the expert system comprises a plurality of rules including each of the following:
a rule responsive to whether fluid flow in the patient is negative;
a rule responsive to whether fluid flow in the patient is neutral;
a rule responsive to whether the patient is taking an antiarrhythmic agent;
a rule responsive to whether to whether the patient has experienced atrial fibrillation;
a rule responsive to an whether a threshold amount of dosages of different classes of pharmaceuticals are candidates to be changed concurrently; and
a rule that adjusts the threshold amount of dosages of different classes of pharmaceuticals based on a blood sugar level of the patient in the information about the current patient condition.

13. The medium of claim 1, wherein the expert system comprises a plurality of rules including each of the following:
a rule that takes a variable;
a rule that modifies a value of a variable in another rule conditional upon inputs;
a rule that outputs a recommendation conditional upon inputs; and
a rule that that outputs a prohibition conditional upon inputs.

14. The medium of claim 1, wherein:
the expert system comprises more than 250 rules;
the prescription is updated more than 5 times for a given patient;
the number of prescription-specific models is greater than or equal to 4; and
the AI model is responsive to more than 10 different lab test measurements and demographic attributes.

15. The medium of claim 1, wherein the expert system comprises rules encoding:
steps for determining a response to a beta negative flow of the patient.

16. The medium of claim 1, wherein the expert system comprises rules encoding:
steps for determining a response to use of sotalol by the patient.

17. The medium of claim 1, wherein the expert system comprises rules encoding:
steps for determining a response to a fluid negative flow of the patient.

18. The medium of claim 1, wherein the AI application is configured to present a recommended updated prescription in a user interface based on output of the expert system.

19. The medium of claim 1, wherein the information about the patient comprises:
impedance cardiograph data based upon variation in impedance measurements during heart beats from an alternating current applied to a set of four or more electrodes placed on the patient adjacent a neck of the patient and a diaphragm of the patient;
age and gender of the patient;
a value indicative of use of a loop diuretic with the patient; and
values indicative of results of at least three of the following tests of the patient:
a measurement of B-type natriuretic peptide (BNP) in blood of the patient,
a measurement of N-terminal pro-BNP in blood of the patient,
a metabolic panel indicative of an amount of electrolyte imbalance, kidney failure, or liver disease of the patient,
a complete blood count (CBC) indicative of anemia,
a thyroid tests indicative of an amount of thyroid hormone in blood of the patient,
a measurement of Galectin-3 protein in blood of the patient, or
a measurement of ST2 protein in blood of the patient.

20. A method, comprising:
obtaining, with an artificial intelligence (AI) application executed by a computer system, information about a current patient condition, wherein:
the information relates to a patient who has undergone heart failure,
at least some of the information is obtained from an electronic medical records system via an application program interface of the electronic medical records system,
at least some of the information is validated or entered into the AI application by a medical professional after obtaining information from the electronic medical records system, and
the AI application comprises:
a plurality of pharmaceutical-specific models each corresponding to a different class of pharmaceuticals to treat heart failure and each configured to predict a response of the patient to a change in dosage of the corresponding class of pharmaceuticals given the current patient condition, and
an expert system comprising a plurality of rules in a rule-graph, the rule graph being traversable to determine an updated prescription based on the patient condition and the predicted patient responses;
inputting, with the computer system, at least some of the obtained information about the patient condition into the respective pharmaceutical-specific models and, in response:
predicting respective changes in the condition of the patient responsive to respective changes in respective dosages of the respective classes of pharmaceuticals to obtain a set of predicted patient responses for the different classes of pharmaceutical,
selecting based on the set of predicted patient responses, candidate classes of pharmaceuticals for an updated prescription,
determining differences between target dosages and the current dosages of the classes of pharmaceuticals, and
based on the differences, determining respective priority scores of the respective classes of pharmaceuticals;
inputting, with the computer system, into the expert system of the AI application, the candidate classes of pharmaceuticals and their respective priority scores and, in response, determining an updated prescription; and
storing, with the computer system, the updated prescription in memory.
